(19) United States
(12) Patent Application Publication
(10) Pub. No.: US 2007/0197543 A1
(43) Pub. Date: Aug. 23, 2007

(54) Use of derivatives of 2, 5-dihydroxyb enzene-sulphonic acids in the elaboration of a medicinal product to enhance the effect of other drugs used for the treatment of erectile dysfunction

(75) Inventors: Jose Esteve-Soler, Barcelona (ES); Inigo Saenz De Tejada-Gorman, Barcelona (ES)

Correspondence Address: OSTROLENK FABER GERB & SOFFEN, 1180 AVENUE OF THE AMERICAS, NEW YORK, NY, 100368403, US

(73) Assignee: Laboratorios del Dr. Esteve, S.A.

(21) Appl. No.: 11/641,269

(22) Filed: Dec. 19, 2006

Related U.S. Application Data
(62) Division of application No. 10/482,457, filed on Mar. 11, 2004, now abandoned.

(30) Foreign Application Priority Data
Jul. 2, 2001 (ES) P0101535

(51) Int. Cl. A61K 31/519; A61K 31/185

(52) U.S. Cl. 514/252.16; 514/262.1; 514/553

(57) ABSTRACT

The present invention refers to the use of derivatives of 2,5-dihidroxybenzenosulphonic acids of general formula (I), to develop medicinal products of therapeutic value to enhance the effects of phosphodiesterase-5 including sildenaphyl, vardenaphyl and IC-351, of apomorphine, of nitric oxide donors including amyl nitrate, nitroglycerine, nitroprussiate, nitrosothioles and nicorandyl, of the compounds that increase the level of cyclic GMP in the penile tissue and of other compounds used to facilitate penile erection in man.

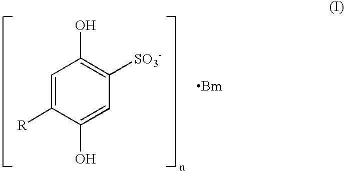

### CROSS REFERENCE TO RELATED APPLICATIONS

This is a divisional of U.S. Ser. No. 10/482,457, filed Mar. 11, 2004, in the name of Jose ESTEVE-SOLER and Inigo SAENZ DE TEJADA-GORMAN, entitled “THE USE OF DERIVATIVES OF 2,5-DIHYDROXYBENZENE-SULPHONIC ACIDS IN THE ELABORATION OF A MEDICINAL PRODUCT TO ENHANCE THE EFFECT OF OTHER DRUGS USED FOR THE TREATMENT OF ERECTILE DYSFUNCTION”, which is a 35 U.S.C. § 371 national phase conversion of PCT/ES02/00325, filed Jul. 1, 2002, which claims priority of Spanish Patent Application No. P0101535, filed Jul. 2, 2001, the entire contents of which is hereby incorporated by reference.

### FIELD OF THE INVENTION

The present invention refers to the use of 2,5-dihydroxybenzenosulphonic acids of general formula (I) in the production of medicinal products of therapeutic value to enhance the effects of phosphodiesterase-5 inhibitors including sildenaphyl, vardenaphyl and IC-351, of apomorphine, nitric acid donors including amyl nitrate, nitroglycerine, nitroprussiate, nitrosothiols and nicorandyl, of compounds that increase cyclic GMP levels in the penile tissue and of other compounds used to facilitate penile erection in man.

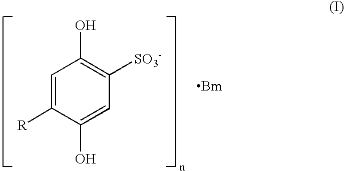

### BRIEF DESCRIPTION OF THE DRAWINGS

FIG. 1 is a graph illustrating the effect of Calcium dobesilate on the relaxation of resistance induced by sodium nitroprussiate in arteries located within the human penis;

FIG. 2 is a graph illustrating the effect of Calcium dobesilate on the relaxation of resistance induced by sildenaphyl in arteries located within the human penis;

FIG. 3 is a graph illustrating the effect of Calcium dobesilate on the relaxation of resistance induced by electrical stimulation in arteries located within the human penis; and

FIG. 4 is a graph illustrating the effect of Calcium dobesilate combined with sildenaphyl on the relaxation of resistance induced by electrical stimulation in arteries located within the human penis.

### DETAILED DESCRIPTION OF THE INVENTION

The present invention refers to the use of derivatives of 2,5 dihydroxybenzenosulphonic acids in the production of drugs of therapeutic value to enhance the effects of phosphodiesterase inhibitors including sildenaphyl, vardenaphyl and IC-351, of apomorphine, of nitric oxide donors including amyl nitrate, nitroglycerine, nitroprussiate, nitrosothioles and nicorandyl, of compounds that increase the level of cyclic GMP in penile tissue and of other compounds used to facilitate penile erection in man.

In recent studies, we have shown that compounds of general formula (I) exert effects on the resistance arteries of the human penis that result in enhancement of the effects of phosphodiesterase-5 inhibitors, such as sildenaphyl, and of apomorphine, of the nitric acid donors and of other products destined to facilitate penile erection in man.

It is known that the therapeutic response to sildenaphyl is variable in different patients and often does not exceed 50% [M S Rendell et al, JAMA 1999, 281: 421-426; R Virag, Urology 1999; 54: 1073-1077], which creates a deficient therapeutic situation.

The compounds referred to in the present invention have general formula (I): 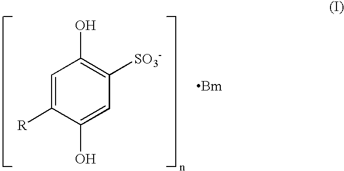
in which:
R represents a hydrogen atom or a sulphonate group (SO₃⁻);
B represents a calcium ion (Ca⁺⁺) or a diethylammonium group [H₂N⁺(C₂H₅)₂];
n represents 1 or 2; and
m represents 1 or 2.

The compounds of the following examples are prepared according to the procedures described previously:

### EXAMPLE 1

Calcium 2,5-dihidroxybenzenosulphonate (Calcium dobesylate). “The Merck Index”, 12 edition, Merck & Co., Whitehorse Station, N.J., USA, 1996.

### EXAMPLE 2

Diethylammonium 2,5-dihidroxybenzenosulphonate (Ethamsylate). “The Merck Index”, 12 edition, Merck & Co., Whitehouse Station, N.J., USA, 1996.

### EXAMPLE 3

Bis-diethylammonium 2,5-dihidroxybenzene-1,4-disulphonate (Bis-diethylammonium persilate). French patent FR 73/17709 (publication number 2.201.888).

To study the enhancing effect of medicinal products used to facilitate penile erection in man a series of studies were carried out of the resistance arteries of the human penis, obtained from patients submitted to penile prosthesis implantation.

Specimens of human cavernous bodies of the penis were obtained from patients with impotence while these were intervened for prosthetic implantation, as described previously (Gupta et al.; Br. J. Pharmacol., 116: 2201, 1995). The tissues were deposited in M-400 solution (pH 7.4; 400 mOsm/kg. Composition in w/v: 4.19% manitole, 0.2% KH₂PO₄, 0.97% K₂HPO₄.3 H₂O, 0.11% KCl and 0.08% NaHCO₃) at 4° C. at the moment of explant and were transported to the laboratory to be used within the following 16 h.

The resistance arteries of the penis, helicine arteries (with a luminal diameter of 150-400 μm), which are terminal branches of the deep arteries of the penis, were dissected carefully removing the surrounding trabecular tissue and were cut into 2 mm long arterial segments that were arranged on two wires of 40 μm diameter in a Halpern-Mulvany myograph (J.P. Trading, Aarhus, Denmark) to record isometric pressure. The cavities contained physiological saline solution (PSS) through which a mixture of 95% O₂/5% CO₂ was continually passed to maintain this oxygenated and to maintain the pH at around 7.4. The arteries were contracted with 1 μM of noradrenaline and their relaxation responses were assessed after adding to the cavities increasing amounts of the different compounds. Transmural electrical stimulation (TES) was carried out using two electrodes placed parallely to the arterial segment and connected to a stimulator with a direct output current (50 mA). Squared pulses were applied of 0.3 ms duration in relays of 15 s with variable frequency (0.5, 1, 2 and 6 Hz).

Effects on the Relaxation of Resistance Arteries of the Human Penis Enhanced by a Specific Nitric Oxide Donor.

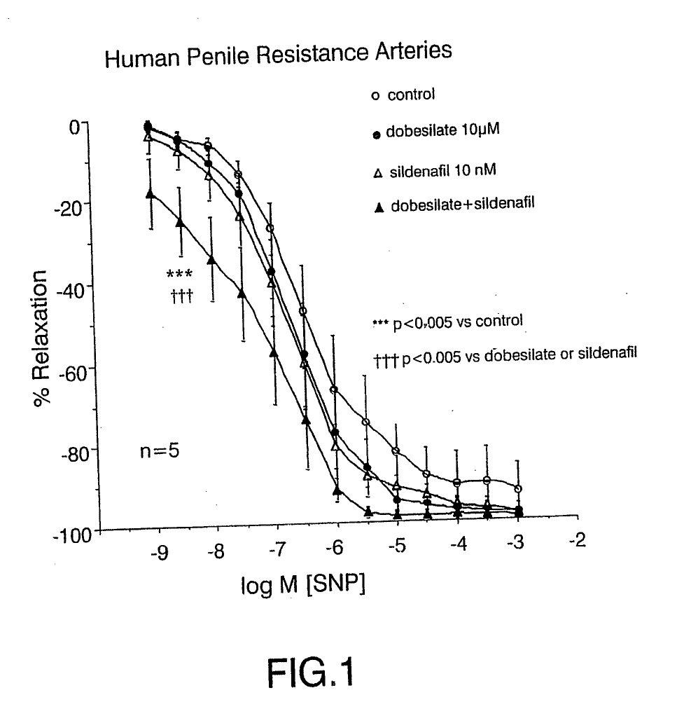

Calcium dobesylate at a concentration of 10 μM increases, in a statistically significant manner, the relaxation produced by different concentrations of sodium nitroprussiate (SNP), a known nitric oxide donor (FIG. 1).

Effects on the Relaxation of Resistance Arteries of the Human Penis Induced by Sildenaphyl.

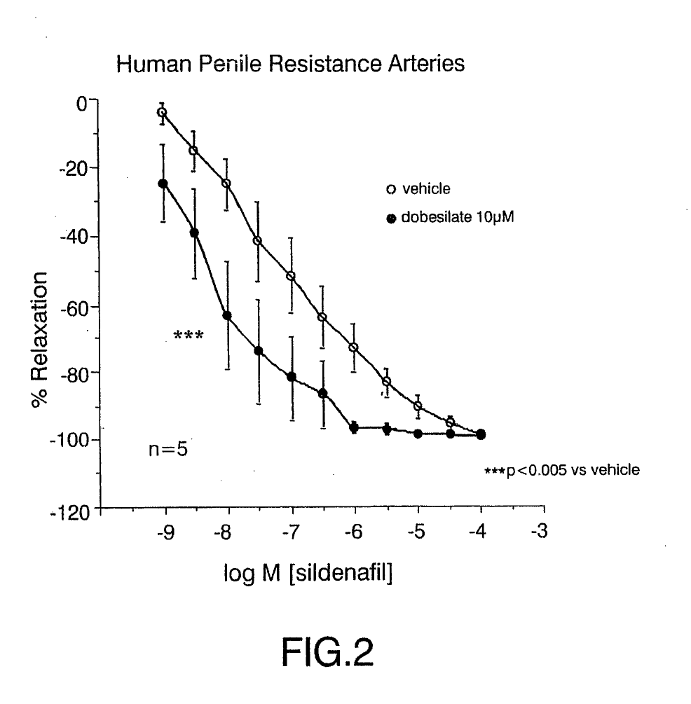

Calcium dobesylate at a concentration of 10 μM increases, in a statistically significant manner, the relaxation produced by different concentrations of the inhibitor of 5-sidenaphyl phospodiesterase (FIG. 2).

Effects on the Relaxation of Resistance Arteries of the Human Penis Induced by Electrical Stimulation of Nitrergic Terminations.

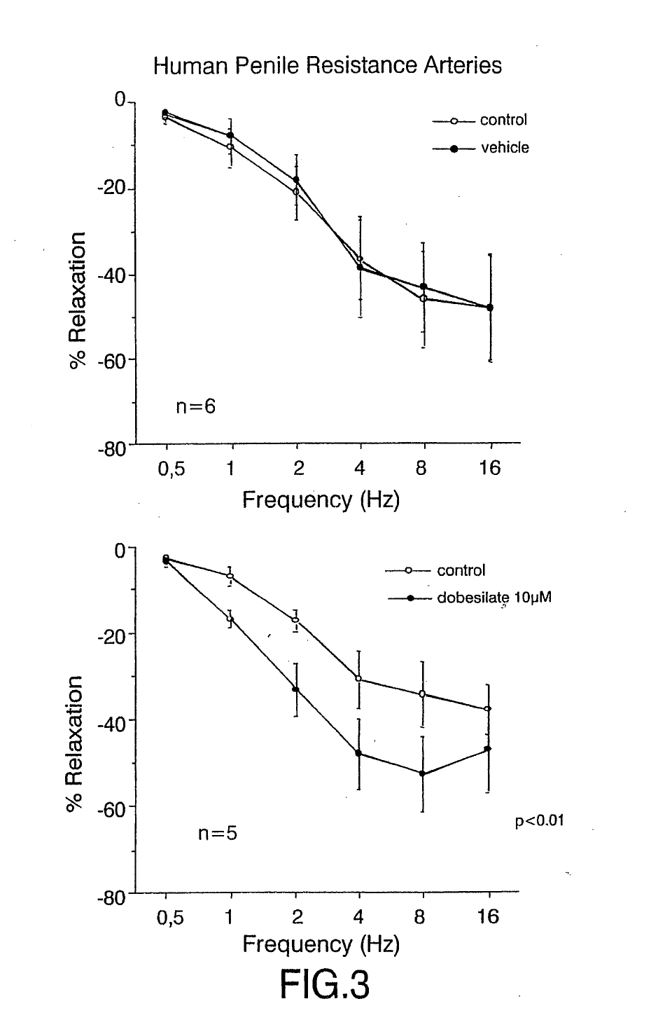

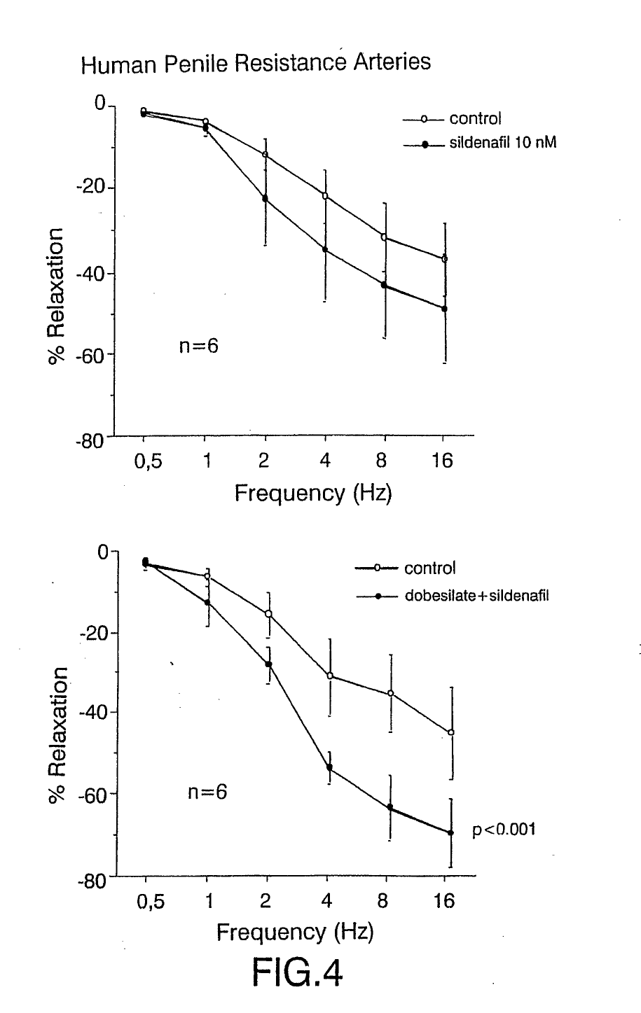

Calcium dobesylate at a concentration of 10 μM increases, in a statistically significant manner, the relaxation produced by electrical stimulation at increasing frequencies of the nitrergic terminations in resistance arteries of the human penis (FIG. 3). This effect is similar and even greater than that produced by sildenaphyl at a concentration of 10 nM (FIG. 4).

Calcium dobesylate, at a concentration of 10 μM, increases, in a statistically significant manner, the effects of 10 nM of sildenaphyl on the relaxation produced by electrical stimulation at increasing frequencies of the nitrergic terminations in resistance arteries of the human penis (FIG. 4).

What is claimed is:

1. A method for enhancing an effect of at least one of a phosphodiesterase-5 inhibitor, apomorphine, a nitric oxide donor, a compound which increases the level of cyclic GMP in penile tissue and any other compound used to facilitate penile erection in man, wherein said method comprises the simultaneous administration, in the same medicament, of said at least one of a phosphodiesterase-5 inhibitor, apomorphine, nitric oxide donor, compound which increases the level of cyclic GMP in penile tissue and any other compound used to facilitate penile erection in man, together with an effective amount of a 2,5-dihydroxybenzenosulphonate acid of general formula I 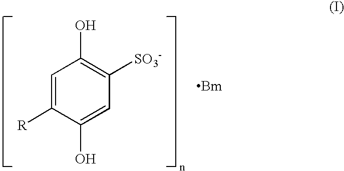
in which
R represents a hydrogen atom or a sulphonate group (SO₃⁻);
B represents a calcium ion (Ca⁺⁺) or a diethylammonium group [H₂N⁺(C₂H₅)₂];
n represents 1 or 2; and
m represents 1 or 2.

2. The method of claim 1, wherein said derivative is 2,5-dihydroxybenzenosulphonate of calcium (calcium dobesylate).

3. The method of claim 1, wherein said derivative is diethylammonium 2,5-dihydroxybenzenosulphonate (Ethamsylate).

4. The method of claim 1, wherein said derivative is bis-diethylammonium 2,5-dihydroxybenzo-1,4-disulphonate (Persylate).
